(19) United States
(12) Patent Application Publication
(10) Pub. No.: US 2010/0035300 A1
(43) Pub. Date: Feb. 11, 2010

(54) Producing a Target Protein Using Intramolecular Cleavage by TEV Protease

(76) Inventors: Andrew H.-J. Wang, Taipei (TW); Ting-Fang Wang, Taipei (TW); Yan-Ping Shih, Taipei (TW); Hui-Chung Wu, Taipei (TW); Su-Ming Hu, Taipei (TW)

Correspondence Address: Luce, Forward, Hamilton & Scripps LLP, 2050 Main Street, Suite 600, Irvine, CA, 92614, US

(21) Appl. No.: 11/364,716

(22) Filed: Feb. 27, 2006

(51) Int. Cl. C12P 21/00; C12N 1/20

(52) U.S. Cl. 435/69.1; 435/252.8

(57) ABSTRACT

A cis-TEVP fusion protein including a TEVP protease, a TEVP cleavage site and a target protein provide a platform for expression of the target protein. A trans-TEVP fusion protein including a TEVP cleavage site and a target protein, the amino-terminal portion of the target protein adjacent to the C-terminal portion of the TEVP cleavage site, the amino acidic residue in position P2 of the TEVP cleavage site being a Valine also produces the target protein by the same process. A cis-TEVP fusion protein system comprising the first fusion protein and a suitable host cell; a trans-TEVP fusion protein system comprising the second fusion protein and a suitable host cell; associated methods to produce target proteins, and kits of parts are also disclosed herein.

### FIELD OF THE DISCLOSURE

The present disclosure relates to production of proteins and in particular to fusion proteins and fusion protein systems.

### BACKGROUND

Production of proteins and in particular of recombinant proteins is a major issue in modern biological research and biotechnological industry.

Fusion protein systems (FPS) have been developed as particularly effective high-throughput systems for producing highly soluble recombinant proteins suitable for functional and structural analysis.

An FPS typically comprises a fusion protein including a passenger or target protein together with a fusion carrier or affinity tag system, and a protease or other suitable enzymes for separating the carrier from the target protein. The protease can be included in the fusion protein (cis-approach) or provided separately (trans-approach).

In the FPS-mediated recombinant proteins production, the target protein is separated from the fusion carrier by site-specific proteolysis, typically performed after affinity chromatography. This has proven to be one of the crucial steps of FPS-mediated recombinant proteins' production, especially in applications such as structural biology or protein-drug production.

Steric hindrance at the cleavage site, optimization of cleavage conditions, high cost of certain proteases, aggregation of cleaved target proteins after removal from their fusion carrier, efficient interaction of affinity tags with their immobilized ligands and presence of extraneous unwanted amino acidic residues due to the introduction of protease-specific recognition sites and/or of restriction cloning sites in the engineered linker region, are reported in the art as the most significant problems affecting FPS-mediated recombinant protein production in vitro.

Efforts have been made to provide FPS with an improved efficiency as to yields and solubility of the produced recombinant proteins. FPS including Factor Xa (FXa) or Tobacco Etch Virus Protease (TEVP) (Sambrook and Russell, 2000) in particular have given good results in overcoming the above problems.

TEVP exhibits high sequence stringency (Dougherty et al. 1989; Phan et al. 2002) and can be overexpressed in E. coli or eukaryotic cells without interfering with cell viability (Kapust and Waugh, 2000; Gruber et al. 2003). TEVP is a protease that specifically cleaves the amino acid sequence -Glu(P6)-P5-P4-Tyr(P3)-P2-Gln(P1)-↓-P1′- in a fusion protein, where P2, P4 and P5 positions are non-conserved amino acids (Dougherty et al. 1989; Kapust et al. 2002).

An intracellular fusion protein processing system wherein TEVP is provided in trans had been developed and exhibited high specificity in processing in E. coli. This system used two compatible expression vectors to separately produce TEVP and a maltose binding protein (MBP) fusion protein containing the TEV recognition site (rsTEV) (Kapust and Waugh, 2000).

However, this intracellular processing system still encounters most problems of the in vitro cleavage methods described above. Additionally, a rather long PCR forward primer is used for addition of the rsTEV protease recognition or cleavage site at 5′-end of the passenger protein gene.

### SUMMARY OF THE DISCLOSURE

According to a first aspect, an FPS is disclosed which includes a TEVP protease and a TEVP cleavage site wherein the TEVP protein is provided in cis (cis-TEVP-FPS). The cis-TEVP-FPS, includes a cis-TEVP fusion protein comprising a TEVP and a TEVP cleavage site, wherein site-specific self-cleavage of the fusion protein at said TEVP cleavage site is detectable upon expression of the fusion protein in a host cell. The cis-FPS can include also a suitable expression vector for the expression of the fusion protein and/or a suitable host cell for the expression of the cis-TEVP fusion protein.

According to a second aspect, a cis-TEVP fusion protein is disclosed, the cis-TEVP fusion protein comprises a TEVP and the TEVP cleavage site, wherein upon expression of the cis-TEVP fusion protein in a suitable host, site-specific self-cleavage of the cis-TEVP fusion protein at said TEVP cleavage site can be detected.

According to a third aspect, a method to produce a target protein is disclosed, the method comprise providing a cis-TEVP fusion protein expression vector including a polynucleotide encoding for a cis-TEVP fusion protein, the cis-TEVP fusion protein comprising a TEV protease, a TEVP cleavage site and a target protein; providing a suitable host cell transformable with the cis-TEVP fusion protein expression vector; transforming a suitable host cell with the cis-TEVP fusion protein expression vector; and obtaining the target protein from the transformed suitable host cell, the target protein obtained upon expression of the cis-TEVP fusion protein in the suitable host, by site-specific self-cleavage of the cis-TEVP fusion protein at said TEVP cleavage site.

According to a fourth aspect a kit of parts for the production of a target protein is disclosed, the kit comprising: at least two among a cis-TEVP fusion protein expression vector, the cis-TEVP fusion protein expression vector including a polynucleotide encoding for a cis-TEVP fusion protein comprising a TEV protease, a TEVP cleavage site and a target protein; a cis-TEVP expression vector, the cis-TEVP expression vector including a polynucleotide encoding for a fusion protein comprising a TEV protease and a TEVP cleavage site, the cis-TEVP expression vector modifiable into the cis-TEVP fusion protein expression vector by introduction in the polynucleotide encoding for a target protein in the TEVP cleavage site; a cis-TEVP construction vector, the cis-TEVP construction vector being a vector modifiable into the cis-TEVP expression vector by introduction of a polynucleotide coding for a TEV protease, a TEVP cleavage site or portions thereof; and a suitable host cell transformable with the cis-TEVP fusion protein expression vector.

The target protein is obtained upon transformation of the host cell with the cis-TEVP fusion protein expression vector, by site-specific self-cleavage of the cis-TEVP fusion protein produced in the host cell at said TEVP cleavage site.

Additional components such as one or more polynucleotides coding for a target protein can also be included in the kit of parts.

According to a fifth aspect, an FPS is disclosed which includes a TEVP protease and a TEVP cleavage site wherein the TEVP protein is provided in trans (trans-TEVP-FPS). In the trans-TEVP-FPS the fusion protein comprises a TEVP cleavage site and a target protein, wherein the amino-terminal portion of the target protein is adjacent to the C-terminal portion of the TEVP cleavage site, the amino acidic residue in position P2 of the TEVP cleavage site is a Valine, and wherein upon expression of the fusion protein in a suitable host cell in presence of a TEVP protease, site-specific cleavage of said fusion protein at said cleavage site can be detected.

The trans-TEVP-FPS can also include a suitable expression vector for the expression of the fusion protein and/or a suitable host cell. Optionally in embodiments wherein the TEVP protease function is not provided by the host cell a TEVP function expression vector providing the TEVP function can be included in the cells.

According to a sixth aspect, a trans-TEVP fusion protein is disclosed, the trans-TEVP fusion protein comprising a TEVP cleavage site and a target protein, wherein the amino-terminal portion of the target protein is adjacent to the C-terminal portion of the TEVP cleavage site, the amino acidic residue in position P2 of the TEVP cleavage site is a Valine, and wherein upon expression of the fusion protein in a suitable host cell in presence of a TEVP protease, site-specific cleavage of said trans-TEVP fusion protein at said cleavage site can be detected.

According to a seventh aspect, a method to produce a target protein is disclosed, the method comprising: providing a trans-TEVP fusion protein expression vector, the trans-TEVP fusion protein expression vector comprising a polynucleotide encoding for a trans-TEVP fusion protein, wherein the trans-TEVP fusion protein comprises a TEVP cleavage site and a target protein, with the amino terminal portion of the target protein adjacent to the C-terminal portion of the TEVP cleavage site and the amino acid residue in position P2 of the TEVP cleavage site being a Valine; providing a TEVP protease expression vector comprising a polynucleotide encoding for a TEV protease; providing a suitable host cell, the host cell transformable by the trans-TEVP fusion protein expression vector and the TEVP protease expression vector; transforming the suitable host cell with the first trans-TEVP fusion protein expression vector and the TEVP protease expression vector; and obtaining the target protein from the transformed cell, the target protein obtained upon expression of the trans-TEVP fusion protein in the suitable host, by site-specific self-cleavage of the trans-TEVP fusion protein at said TEVP cleavage site.

The portion of the polynucleotide encoding the TEVP cleavage site in the trans-TEVP fusion protein preferably includes the site for SnaBI.

According to an eighth aspect, a method to produce a target protein is disclosed, the method comprising providing the above mentioned trans-TEVP fusion protein expression vector; providing a suitable host cell, the host cell able to express a TEV protease and being transformable by the trans-TEVP fusion protein expression vector; transforming the suitable host cell with the trans-TEVP expression vector; and obtaining the target protein from the transformed cell upon expression of the TEV protease, the target protein obtained upon expression of the trans-TEVP fusion protein in the suitable host, by site-specific self-cleavage of the trans-TEVP fusion protein at said TEVP cleavage site.

According to a ninth aspect, a kit of parts is disclosed, the kit of parts for the production of a target protein the kit comprising at least two among: a trans-TEVP fusion protein expression vector, the trans-TEVP fusion protein expression vector including a polynucleotide encoding for a trans-TEVP fusion protein, the trans-TEVP fusion protein comprising a TEVP cleavage site, the TEVP cleavage site including a Valine amino acidic residue in position P2, and a target protein; a trans-TEVP fusion protein expression vector, the trans-TEVP fusion protein expression vector comprising a TEVP cleavage site, the TEVP cleavage site having a Valine amino acid residue in position P2, the trans-TEVP expression vector modifiable into the trans-TEVP fusion protein expression vector by introduction in the polynucleotide encoding for a target protein in the TEVP cleavage site; a trans-TEVP construction vector, the trans-TEVP construction vector being a vector modifiable into the trans-TEVP expression vector by introduction of a polynucleotide coding for a TEVP cleavage site or portions thereof, the TEVP cleavage site including a Valine amino acidic residue in position P2; a host cell transformable with the trans-TEVP fusion protein expression vector, wherein the target protein is obtained in the suitable host by site-specific self-cleavage of the fusion protein at said TEVP cleavage site, the trans-TEVP fusion protein expression vector and the host cell thereby enabling the production of the target protein.

The kit can further include additional components such as one or more of polynucleotides coding for one or more target proteins.

The fusion protein expression vector and the host cell can be used in the production of a target protein according to the methods herein disclosed.

According to a tenth aspect, a polynucleotide is disclosed, the polynucleotide encoding for at least one of the above-mentioned fusion proteins or a portion thereof.

In particular, according to an eleventh aspect, a polynucleotide is disclosed, the polynucleotide encoding a fusion protein comprising a TEVP cleavage site, wherein the amino acidic residue in position P2 of the TEVP cleavage site is a Valine or a portion thereof. In the polynucleotide, the portion coding for the TEVP cleavage site includes a SnaBI cleavage site.

According to a twelfth aspect, an expression vector is disclosed, the expression vector comprising at least one of the above mentioned polynucleotides or a portion thereof.

In particular, according to a thirteenth aspect, a vector is disclosed comprising the polynucleotide wherein the portion coding for the TEVP cleavage site includes a SnaBI cleavage site.

According to a fourteenth aspect, a cell is disclosed, the cell including at least one of the above-mentioned fusion proteins, polynucleotides and expression vectors.

### BRIEF DESCRIPTION OF THE DRAWINGS

The above-mentioned features and objects of the present disclosure will become more apparent with reference to the following description taken in conjunction with the accompanying drawings in which:

FIG. 1 shows a schematic map of the MBP-TEVP portion of an MBP-TEVP fusion protein expression vector; MBP indicated the portion of the vector coding for Maltose Biding Protein; TEVP indicated the portion of the vector coding for the TEV Protease;

FIG. 2 shows a schematic map of the MBP-TEVP-rsTEV-EGFP-His₆ portion of the MBP-TEVP-rsTEV-EGFP-His₆ fusion protein; the wording “MBP” indicates the portion including the Maltose Biding Protein; the wording “TEVP” indicates the portion including the TEV Protease; the wording “rsTEV” indicates the portion including the TEV protease recognition site; the wording “EGFP” indicates the portion including the Green Fluorescent Protein; the wording “His₆” indicates the portion including the Hexahistidin tag;

FIG. 3A shows the results of an SDS-PAGE performed upon samples of the total protein and soluble protein fractions of cells expressing MBP-TEVP-rsTEV-EGFP-His₆ fusion protein expression vector; lanes 1 and 4 show the whole cell lysates of E. coli cells induced with IPTG; lanes 2 and 5 show whole cell lysates of uninduced E. coli cells; lanes 3 and 6 show soluble proteins from IPTG-induced E. coli cells; the molecular weight standards are shown on the left; the expression of MBP-TEVP is indicated on the right; expression of EGFP-His₆ protein is marked by an asterisk and indicated on the right;

FIG. 3B shows the results of a Western Blot analysis performed on the soluble protein fractions from IPTG-induced E. coli cells of lanes 3 and 6 of FIG. 3A, using anti-His₆ antibody to confirm expression of EGFP-His₆ protein; expression of EGFP-His₆ protein is indicated by a bar and the wording EGFP-His₆ on the left;

FIG. 4 shows the visualization of EGFP-His₆ in IPTG induced E. coli cells; images of living cells expressing MBP-TEVP-rsTEV-EGFP-His₆ fusion protein expression vector (squares A and C) and of living cells expressing MBP-TEVP fusion protein expression vector (squares B and D) were taken by a fluorescence microscopy using either UV light (squares A and B) or visible light (squares C and D);

FIG. 5A shows the results of an SDS-PAGE performed upon samples of the total protein and soluble protein fractions of cells expressing MBP-TEVP-rsTEV-Sso1889-His₆ fusion protein expression vector; lane 7 shows the whole cell lysates of E. coli cells induced with IPTG; lane 8 shows the whole cell lysates of uninduced E. coli cells; lane 9 shows the soluble proteins from IPTG-induced E. coli cells; the molecular weight standards are shown on the left; Sso1889-His₆ is indicated and marked by a bar on the right; the MBP-TEVP-rsTEV protein bands are indicated and marked by a bar on the right;

FIG. 5B shows the results of a Western Blot analysis performed on the soluble protein fractions from IPTG-induced cells of lane 9 of FIG. 5A, performed using anti-His₆ antibody to confirm expression of Sso1889-His₆; the Sso1889-His₆ protein is indicated and marked by a bar on the left;

FIG. 6 shows a schematic representation of an rsTEVP portion of the fusion protein expression vector including an SnaBI cleavage site; from the bottom to the top: the nucleotidic sequence, the related amino acidic sequence and the positions of the amino acid residues with respect to the cleavage site are indicated;

FIG. 7 shows a schematic representation of the sticky-end PCR cloning strategy used to any target protein gene into MBP-TEV expression vector; the codon and anticodon of the amino acid residues in the P1′ position are indicated as ‘XXX’ or ‘YYY’, respectively;

FIG. 8 shows a schematic representation of an MBP-TEVP-rsTEV-EGFP-His₆ fusion protein coded by a fusion protein vector including an SnaBI cleavage site construed according to the cloning strategy illustrated in FIG. 7; the wording “MBP” indicates the portion including the Maltose Biding Protein; the wording “TEVP” indicates the portion including the TEV Protease; the wording “ENLYVQZ” indicates the amino acid residues composing the TEV protease recognition site, wherein Z indicates the amino acid residue in P1′ position; the wording “EGFP” indicates the portion including the Green Fluorescent Protein; the wording “His₆” indicates the portion including the Hexahistidin tag;

FIG. 9A shows the results of an SDS-PAGE performed upon samples of soluble protein lysates from IPTG induced E. coli cells producing MBP-TEVP-rsTEV-EGFP-His₆ having the structure shown in FIG. 8, wherein the amino acidic residue Z in position P1′ is Glycine, Methionine, Valine or Proline as indicated in the figure by the single letter code G, M, V and P respectively; lanes 1, 4, 7 and 10 show the whole cell lysates of E. coli cells induced with IPTG; lanes 2, 5, 8 and 11 show the whole cell lysates of uninduced E. coli cells; lanes 3, 6, 9 and 12 show the soluble proteins from IPTG-induced E. coli cells; the expression of MBP-TEVP-rsTEV-EGFP-His₆, MBP-TEVP-rsTEV and EGFP-His6 protein bands is marked by arrows on the right; the molecular weight standards are shown on the left;

FIG. 9B shows the results of a Western Blot analysis performed on the soluble protein fractions from IPTG-induced cells of lanes 3, 6, 9 and 12 of FIG. 9A, using anti-His₆ antibody to verify expression of EGFP-His₆; the expression of MBP-TEVP-rsTEV-EGFP-His₆, MBP-TEVP-rsTEV and EGFP-His₆ protein bands is marked by arrows and indicated on the right;

FIG. 10 shows a schematic representation of an FC-TEVP-rsTEV-EGFP-His₆ fusion protein coded by a fusion protein vector including an SnaBI cleavage site; the wording “FC” indicates the portion including a fusion carrier; the wording “TEVP” indicates the portion including the TEV Protease; the wording “ENLYVQG” indicates the amino acid residues composing the TEV protease recognition site; the wording “EGFP” indicates the portion including the Green Fluorescent Protein; the wording “His₆” indicates the portion including the Hexahistidin tag;

FIG. 11 shows the results of an SDS-PAGE performed upon samples of soluble protein lysates from IPTG induced E. coli cells producing the FC-TEVP-rsTEV-EGFP-His₆ construct of FIG. 10, wherein the FC portion includes with NusA, MBP, GST, Trx, CBP or His₆ as a fusion carrier; lanes 1, 4, 7, 10, 13 and 16, show total cell lysates of E. coli cells induced with IPTG; lanes 2, 5, 8, 11, 14 and 17, show total cell lysates of uninduced E. coli cells; lanes 3, 6, 9, 12, 15, and 18 show the soluble proteins from IPTG-induced E. coli cells; the expression of cleaved products NusA-TEVP-rsTEV, MBP-TEVP-rsTEV, GST-TEVP-rsTEV, Trx-TEVP-rsTEV, CBP-TEVP-rsTEV, EGFP-His₆ and His₆-TEVP-rsTEV is marked by arrowheads and also indicated on the left; and

FIG. 12 shows the results of a Western Blot analysis performed on the soluble protein fractions from IPTG-induced cells of lanes 3, 6, 9, 12, 15, and 18 of FIG. 11, performed using anti-His₆ antibody to verify expression EGFP-His₆; the expression of cleaved products NusA-TEVP-rsTEV, Trx-TEVP-rsTEV, and EGFP-His₆ is indicated and marked by bars on the right; the molecular weight standards are shown on the left.

### DETAILED DESCRIPTION OF PREFERRED EMBODIMENTS

Different fusion protein systems (FPS) including a TEV protease and a TEVP cleavage site are herein disclosed, the FPS allowing production of a recombinant protein included in the FPS as target or passenger protein.

The phrase “fusion protein” refers to a polypeptide comprising at least two portions having different coding sequences according to the genetic code, e.g. coding sequences from different genes, the at least two portions acting as a single polypeptide. Fusion proteins can be “hybrid proteins.” or “chimeric proteins,” wherein the phrase “chimeric protein” refers to coding sequences that are obtained from different species of organisms, as well as coding sequences that are obtained from the same species of organisms.

The term “protein” or “polypeptide” refers to a polymer of any length and dimensions including a sequence of joined amino acids, such as a covalently-linked sequence of amino acids in which the amino terminal and the carboxy terminal ends on the amino acids are joined by peptide bonds. The phrase “recombinant protein” refers to a protein coded by a combination of at least two coding sequences not found together in a single biological material; exemplary recombinant proteins include a protein coded by DNA formed by bringing together DNA fragments from different species. The phrase “biological material” refers to any material able of self-replication under appropriate condition, such as viruses, eukaryotic or prokaryotic cells, unicellular or multicellular organisms, and other material identifiable by a person skilled in the art. The term “organisms” refers to an individual exhibiting living characteristics composed of one cell or more. The term “species” refers to a group of actually or potentially interbreeding natural populations which are reproductively isolated from other such groups.

The phrase “TEV protease” (TEVP) refers to the tobacco etch virus protease, or a polypeptide functionally equivalent thereto; exemplary TEVPs include wild type tobacco etch virus protease, mutants of the tobacco etch virus protease and recombinant proteins including at least one sequence from wild type tobacco etch virus protease, each of said wild type, mutants, and recombinant proteins, able to provide the cleavage functions associated with the tobacco etch virus protease.

The phrase “TEVP cleavage site” refers to a sequence in a polypeptide that can be recognized and specifically cut by a TEVP, such as the site having the sequence Glu(P6)-P5-P4-Tyr(P3)-P2-Gln(P1)-↓-P1′- or any other site functionally equivalent to Glu(P6)-P5-P4-Tyr(P3)-P2-Gln(P1)-↓-P1′-. Generally the phrase “cleavage site” refers to a sequence in a polynucleotide or polypeptide that can be processed by site-specific proteolysis performed by a specific enzyme.

The positions P6 to P1 and P1′ to P6′ in a cleavage site indicate positions of amino acid residues with respect to an enzyme cleavage site, wherein P6 to P1 indicate sequential positions of the amino acid residues upstream of the cleavage site in the amino-terminal-to-carboxy-terminal direction, and P1′ to P6′ indicate sequential positions of the amino acid residues downstream of the cleavage site in the amino-terminal-to-carboxy-terminal direction; with positions P1 and P1′ adjoining the cleavage site and positions P6 and P6′ removed from the cleavage site.

The phrases “target protein” “passenger protein” or “protein of interest” refer to a protein, e.g. a recombinant protein, whose expression is desired; in a fusion protein, the protein of interest is generally joined or fused with one or more protein or protein domains, also named fusion partner(s), to allow for enhanced stability of the protein of interest and/or ease of purification of the fusion protein.

In particular, FPSs are disclosed that can be used for the production of a recombinant target protein according to a cis-approach or according to a trans-approach.

In one embodiment, production of the target protein is performed following a cis-approach by using cis-TEVP fusion protein system (cis-TEVP-FPS). In the cis-TEVP-FPS, the target protein, TEVP and TVP cleavage site are included in a single fusion protein (cis-TEVP-FPS fusion protein). The cis-FPS can include also a suitable expression vector for the expression of the cis-TEVP-FPS fusion protein and/or a suitable host cell.

The phrase “expression vector” refers to any vector suitable to introduce foreign material including genetic coded information into a suitable host cell in order to have the genetic coded information converted into protein products; in the fusion protein vector the protein products comprise the fusion protein. The phrase “host cell” and the term “host” refer to a prokaryotic or eukaryotic cell which is used to receive, maintain, allow reproduction and/or allow expression of a protein in vitro, i.e. in an artificial environment outside an organism; or in vivo, i.e. within an organism; in particular in the cis-TEVP-FPS, a “suitable host” or a “suitable host cell” is a prokaryotic or eukaryotic cell used to receive, maintain, allow reproduction and/or allow expression of fusion protein, such as a cis-TEVP-FPS fusion protein alone or included in a vector, such as an expression vector.

In the cis-TEVP-FPS, upon expression of the cis-TEVP-FPS fusion protein in the suitable host, site-specific self-cleavage of the cis-TEVP-FPS fusion protein at said TEVP cleavage site, and associated production of the target protein, can be detected.

In the cis-TEVP-FPS fusion protein, the TEV protease is preferably contiguous to the portion including the TEVP cleavage site. More preferably, in the single fusion protein, the TEV protease is located so that the carboxy-terminal portion of the TEVP is adjacent the amino-terminal portion of the TEVP cleavage site.

In one embodiment, the cis-TEVP-FPS fusion protein has the formula

[X1]-[Y]-[L]-[X2] (I)

wherein, X1 is a polypeptide comprising at least one portion for the expression and/or solubilization of the fusion protein; Y is a polypeptide comprising a TEVP protease; L is a linker polypeptide comprising a TEVP cleavage site; and X2 is a polypeptide comprising at least one target protein.

The term “express” or “expression” refers to a process by which a protein comes into existence in a cell, for example, when the protein results from a gene's coded information, the process by which the information is converted into the protein, for example, upon introduction in a host cell of an expression vector; a portion for the expression of the fusion protein is able to initiate or enhance the expression of the fusion protein in a cell. The term “solubilization” or “solubility” refers to the quality or property of a compound, and in particular the fusion protein or portions thereof, of being soluble and to its relative ability of being dissolved; a portion for the solubilization of the fusion protein is able to make soluble or to increase solubility of the fusion protein.

X1 can comprise a carrier or fusion carrier alone or together with additional polypeptides. The phrase “fusion carrier” and the term “carrier” as included in a fusion protein refer to any polypeptide suitable to increase the expression of the fusion protein or of a portion thereof, improve the solubility of the fusion protein or a portion thereof, improve folding/stability of the fusion protein or a portion thereof, increase enzymatic activities associated with the fusion proteins or a portion thereof, minimize formation of inclusion bodies in the process for production of the fusion protein or a portion thereof, allow the fusion protein to be directed to specific cellular compartments, protect the fusion proteins or a portion thereof from proteolysis other than the proteolysis performed by the TEVP in a host cell, tag the fusion protein or a portion thereof and/or isolate the fusion protein or a portion thereof.

In particular, in the cis-TEVP-FPS fusion protein the fusion carrier can provide at least one of the following functions: higher protein solubility, better folding/stability, higher enzymatic activity, and higher protein expression level, compared with the ones of the cis-TEVP-FPS fusion protein in absence of the fusion carrier, and/or can provide an accurate targeting to the desired sub-cellular location.

Fusion carriers which are able to tag the cis-TEVP-FPS fusion protein or portions thereof can be also used as affinity tags in the fusion protein, to allow purification of the fusion protein from the host cell or culture supernatant, or both. The phrase “affinity tag” refers to structures or compounds, such as short amino acid sequences, usually engineered onto the N- or C-terminus of a protein, to make the purification of the protein easier; exemplary affinity tags are a “poly-histidine tract” or “poly-histidine tag,” which facilitate the purification of a recombinant fusion protein from a host cell, host cell culture supernatant, or both; for example, the phrase “poly-histidine tract” and “poly-histidine tag,” when used in reference to a fusion protein, refers to the presence of two to ten histidine residues at either the amino- or carboxy-terminus of a protein of interest, and in particular a poly-histidine tract of six to ten residues; a poly-histidine tract can also be defined functionally as being a number of consecutive histidine residues added to the protein of interest which allows the affinity purification of the resulting fusion protein on a nickel-chelate or IDA column.

The choice of the fusion carriers can depend on the host cells according to parameters identifiable by a person skilled in the art upon reading of the present disclosure. Exemplary fusion carriers that can be included in the cis-TEVP-FPS fusion protein comprise MBP, NusA, thioredoxin (Trx), glutathione S-transferase (GST), calmodulin binding protein (CBP), and His₆ tag. FPSs including the above carriers successfully carried out intracellular detectable site-specific cleavage at the TEVP cleavage site as exemplified in Examples 1 and 4.

Y is a polypeptide that comprises a TEV protease alone or together with an additional polypeptide. In particular the TEV protease can be the TEV protease identified by the Genbank accession number M11458, nucleotide sequence 5691-6980, which was also used in experimental procedures exemplified in the Examples section.

L is a polypeptide linker. The term “linker” refers to a portion of a polynucleotide or polypeptide that contains one or more cleavage sites and is generally placed into a polynucleotide or polypeptide structure, such as a vector or a fusion protein, so that the site/sites may subsequently be used for processing and/or insertion of another polypeptide or polynucleotide. In particular, L is a linker that comprises a TEVP cleavage site alone or together with an additional polypeptide.

Preferably, the TEVP cleavage site included in L is the polypeptide Glu(P6)-XaaP5-XaaP4-Tyr(P3)-ValP2-Gln(P1)-1-XaaP1′- (SEQ ID NO: 1), wherein the amino acid residue in the P2 position of the TEVP cleavage site of Glu(P6)-P5-P4-Tyr(P3)-P2-Gln(P1)-↓-P1′ has been replaced by a Val.

The cis-TEVP-FPS fusion protein including the TEVP cleavage site of SEQ ID NO: 1 is preferably able to carry out up to near 100% site-specific self-cleavage and generates carrier protein-TEVP and target protein constructs with large quantity and high solubility, as shown by experimental procedure exemplified in the Examples section, in particular Example 3. Also, the cis-TEVP-FPS fusion protein including the TEVP cleavage site of SEQ ID NO: 1 is able to carry out site-specific self-cleavage and to generate large quantity of high soluble target protein having any amino acidic residue in their amino-terminal position, as shown by experimental procedure exemplified in the Examples section, in particular in Example 3.

Accordingly, the cis-TEVP-FPS fusion protein including the TEVP cleavage site of SEQ ID NO: 1 allows the production of target proteins with their native amino termini, which is particularly advantageous for production of protein whose amino termini have an essential structural or functional role.

X2 is a polypeptide comprising the target protein alone or together with an additional polypeptide. The target protein can be any polypeptide of any length and dimensions, including proteins coded by genes or by any synthetic coding sequence. Experiments exemplified in Examples 1 and 2 show exemplary embodiments wherein the target protein EGFP (Example 1) and Sso1889 (Example 2) have been successfully produced in a soluble form.

The X2 portion can include more than one target protein. The X2 portion can optionally include an affinity tag, such as His₆, as shown in the exemplary embodiments exemplified in Examples 1 to 4. An affinity tag can be in particular introduced in FPS wherein polypeptide will not be used for clinical purposes.

The X2 portion of the fusion protein can further include additional polypeptides of any sequence and dimensions, according to the design of the user. For example, the X2 portion can include additional affinity tags added to the X2 portion to facilitate protein purification. In general, additional polypeptides can also be included in the X1, L and/or Y portions, according to the design of the user. Additional polypeptides may be included in each of said portions, the additional polypeptide associated with specific purposes identifiable by a person skilled in the art upon reading of the present disclosure in view of criteria such as the host wherein the cis-TEVP-FPS will be produced and the target protein to be produced, the amount of target protein to be produced. Thus, a spacer polypeptide can be added between TEVP and FPS to increase their relative flexibility and to improve TEV cleavage efficiency.

The cis-TEVP-FPS and/or the cis-TEVP-FPS fusion protein can be used to produce a target protein in vitro or in vivo, according to procedures herein described and/or identifiable by a person skilled in the art upon reading of the present disclosure and in particular the Example section; those procedures may also include isolation of the fusion protein from the host and/or removal of the target protein from the fusion protein by a variety of enzymatic or chemical means identifiable by a person skilled in the art.

For example, the cis-TEVP-FPS fusion protein can be expressed in a cell culture, using procedures exemplified in the Examples section and/or procedures identifiable by a person skilled in the art upon reading of the present disclosure. The phrase “cell culture” refers to the in vitro (i.e., outside of the body, such as in a test tube or vat) propagation of cells isolated from living pluricellular organisms, including continuous cell lines (e.g., with an immortal phenotype), primary cell cultures, finite cell lines (e.g., non-transformed cells), and any other cell population maintained in vitro.

In some embodiments the cis-TEVP-FPS can be used to produce the target protein following a method comprising: providing a cis-TEVP fusion protein expression vector including a polynucleotide encoding for a fusion protein, the fusion protein comprising a TEV protease, a TEVP cleavage site and a target protein; providing a suitable host cell transformable with the expression vector; transforming the suitable host cell with the expression vector; and obtaining the target protein from the transformed suitable host cell, the target protein obtained upon expression of the fusion protein in the suitable host, by site-specific self-cleavage of the fusion protein at said TEVP cleavage site.

In particular, providing a cis-TEVP fusion protein expression vector including a polynucleotide encoding for a fusion protein comprising a TEV protease, a TEVP cleavage site and a target protein can be performed by providing a cis-TEVP-FPS vector.

In particular, the cis-TEVP-FPS vector can be an expression vector including a polynucleotide coding for the cis-TEVP-FPS fusion protein (cis-TEVP-FPS polynucleotide). The term “polynucleotide” refers to a polymer of any length and dimension including a sequence of joined nucleotides, such as a covalently-linked sequence of nucleotides in which the 3′ and 5′ ends on the nucleotides are joined by phosphodiester bonds.

The cis-TEVP-FPS polynucleotide, preferably has the following formula

[X1′]-[Y′]-[L′]-[X2′] (II)

where X1′ is a polynucleotide coding for polypeptide X1, Y′ is a polynucleotide coding for polypeptide Y, L′ is a polynucleotide coding for polypeptide L and X2′ is a polynucleotide coding for polypeptide X2.

Preferably, the L′ polynucleotide includes the sequence 5′TACGTA3′ in a position such that the sequence 5′TACGTA3′ codes for the amino acid residue in positions P3 and P2 of the TEVP cleavage site. The cis-TEVP-FPS vector according to this preferred embodiment can be advantageously produced by the high-throughput cloning strategies herein described and exemplified in Example 3. A spacer polypeptide between TEVP and FPS may be included to increase the cleavage efficiency.

The cis-TEVP-FPS vector can contain at least five components: a proper promoter, a translation initiation signal, a target gene cDNA, a transcription terminator, a translational terminator. The cis-TEVP-FPS vector may also include a DNA replication origin of the host cells.

In some embodiments providing an expression vector such as the cis-TEVP-FPS vector can be performed by providing a cis-TEVP expression vector, the TEVP expression vector including a polynucleotide coding for a TEVP and a TEVP cleavage site; providing a polynucleotide coding for a target protein and modifying the cis-TEVP expression vector to include the polynucleotide coding for a target protein, thus obtaining the cis-TEVP-FPS vector.

In some embodiments providing a cis-TEVP expression vector can be performed by providing a cis-TEVP construction vector, wherein the cis-TEVP construction vector is an expression vector modifiable to enclose a polynucleotide coding for a TEVP protease and TEVP cleavage site and/or a part thereof, and modifying the cis-TEVP construction vector to enclose a polynucleotide coding for a TEVP protease and TEVP cleavage site and/or a part thereof, the modified cis-TEVP construction vector being a cis-TEVP expression vector.

In some embodiments the above-mentioned operations can be grouped or be all performed simultaneously.

In particular, in some embodiments, the cis-TEVP-FPS vector can be construed starting from a first cis-TEVP construction vector which is an expression vector including a first cis-TEVP construction polynucleotide coding for one or more of the polypeptides X1, Y and L or portion thereof.

Preferably, the first cis-TEVP construction polynucleotide codes for a first cis-TEVP construction protein which includes a TEVP, a TEVP cleavage site and optionally any desired carrier and/or tag system, the first cis-TEVP construction polynucleotide further including a restriction enzyme cleavage site adjacent the 3′ end of the polynucleotide portion coding for the TEVP cleavage site.

In some embodiments, the first cis-TEVP construction polynucleotide has the formula:

[X1′]-[Y′]-[L′] (III)

wherein the X1′, Y′ and L′ have the same meaning reported for formula (II) and L′ includes a cleavage site in the portion coding for the TEVP cleavage site for introducing of a polynucleotide coding for one or more target proteins.

In some embodiments, the cleavage site is the one for the enzyme SnaBI and includes the SnaBI cleavage site of sequence 5′TACGTA3′, located in the polynucleotide so that 5′TACGTA3′ provides the coding portion for the amino acid residues in positions P3 and P2 of the TEVP cleavage site in the fusion protein.

The first cis-TEVP construction vector can be modified by introducing into the cleavage site, provided in the L′ portion, a polynucleotide coding for a pre-selected target protein thus obtaining the cis-TEVP-FPS fusion protein vector. The modification can be performed by techniques known to the person skilled in the art and exemplified in the Examples section, which will not be thus further described in detail.

In another embodiment the cis-TEVP-FPS vector can be construed starting from a second cis-TEVP construction vector, which is an expression vector including a second cis-TEVP construction polynucleotide coding for portions X1 and Y or a part thereof, such as the MBP-TEV polynucleotide of the expression vector described in Example 1.

In some embodiments, the second cis-TEVP construction polynucleotide has the formula

[X1′]-[Y′] (IV)

wherein the X1′ and Y′ have the same meaning reported for formula II.

According to a cloning strategy, the cis-TEVP-FPS vector is construed starting from the second cis-TEVP construction vector by modifying the second cis-TEVP construction vector to obtain the first construction vector and modifying the first cis-TEVP construction vector to obtain the cis-TEVP-FPS vector.

The second construction vector can be in particular modified to provide the first construction vector, by a sticky-end PCR method of the high-throughput cloning strategies schematically shown in FIG. 7 and exemplified in Example 3 to introduce the polynucleotide L′ including restriction sites suitable for cloning an additional polynucleotide in the vector. In one embodiment, the polynucleotide L′ comprises the SnaBI restriction enzyme site 5′TACGTA3′.

The sticky-end PCR method is performed using three PCR primers (one forward and two reverse) and reactions in two separate tubes. The three primers have the following sequences: forward primer (herein also named primer A): 5′ GTACAGXXXOOOPPPQQQSSSTTT (SEQ ID NO: 2) wherein XXX, OOO, PPP, QQQ, RRR, SSS, TTT represent the genetic codons of the first six amino acids in the target protein, respectively; Reverse primer 1 (herein also named primer C): 5′ TCGAGxxxyyyooopppqqqsssttt (SEQ ID NO: 3), and Reverse primer 2 (herein also named primer D): 5′ Gxxxyyyooopppqqqsssttt, wherein xxx represents the stop codon; yyy,ooo,ppp,qqq,sss,ttt represents the genetic codon of the last six amino acids in the target protein, respectively.

The two PCR reactions are carried out using the primer A and primer C, and primer A and primer D, respectively. The sticky-end PCR method can be performed in such a way that the shortest primers for producing native protein are used. The second cis-TEVP construction vector so obtained can be modified to include a polynucleotide coding for a target protein, following procedure herein described in the Examples section or identifiable by a person skilled in the art upon reading of the present disclosure.

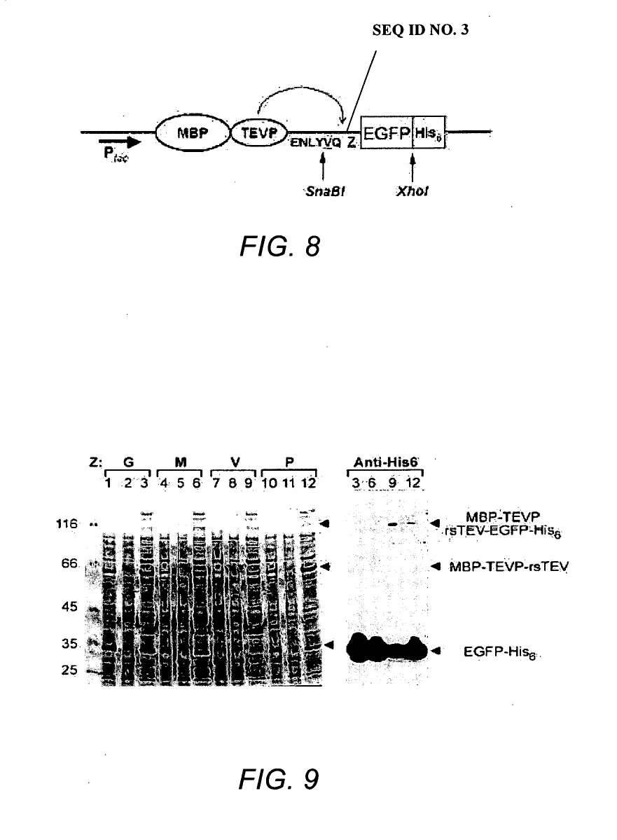

The resulting cis-TEVP-FPS vector because of the Val in the P2 position of the TEVP cleavage site allows the production of target proteins with any amino-termini, which include target proteins with their native amino terminus, as illustrated in FIG. 8 (where Z represents the P1′ amino acid) and exemplified in Example 3, wherein exemplary embodiments of the preferred cloning strategy are also described.

Transforming the suitable host cell with the cis-TEVP-FPS vector and obtaining the target protein from the transformed suitable host cells, can then be performed starting by the cis-TEVP-FPS vector according to various methods to produce a recombinant protein in host cells, such as E. coli, as well as any host cells that are available for in vitro cloning can be used with this approach.

For example, transforming the suitable host cells and obtaining the target protein can be performed by techniques exemplified in the Examples section. A person skilled in the art can identify any additional or alternative procedures, as well as any additional or alternative transcription/translation system to produce the cis-TEVP-FPS vector upon reading of the present disclosure, in particular the Examples section.

Also a person skilled in the art can envision additional procedures to produce the target protein from the cis-TEVP-FPS fusion protein in vitro or in vivo which do not necessarily require the use of expression vectors, upon reading of the present disclosure and in particular the Examples section, based on the target protein to be produced. For example, the cis-TEVP-FPS fusion protein may be introduced into the genome of a host under control of elements regulating the expression of the cis-TEVP-FPS fusion protein.

According to a further aspect, a kit of part, for production of a target protein are also disclosed, the kit can comprise at least two among a cis-TEVP fusion protein expression vector, such as a cis-TEVP-FPS vector, a cis-TEVP expression vector, a cis-TEVP construction vector, such as the first cis-TEVP construction vector and/or the second cis-TEVP construction vector, a polynucleotide coding for the target protein and a suitable host cell.

In embodiments wherein a TEVP construction vector such as the first and/or second construction vector is included, the TEVP construction vector can be modified according to one of the methods disclosed herein to include the polynucleotide coding for the TEVP, the TEVP cleavage site or portions thereof, to obtain the TEVP expression vector, and to further include the target protein to obtain the TEVP fusion protein expression vectors, wherein the host cells can be transformed by the TEVP fusion protein expression vector, thereby enabling the production of the target protein.

In embodiments wherein the cis-TEVP-FPS vector is included, the host cells can be transformed by the cis-TEVP-FPS vector, the cis-TEVP-FPS vector, and the suitable host cell, thereby enabling the production of the target protein. The term “transform” refers to a process in which foreign material is introduced into a suitable or competent host recipient cell, which includes the direct transfer of genetic material from donor to recipient, and the acquisition (e.g., by bacteria cells) of new genetic markers (new traits coded for by the new DNA) via the process of transformation.

The cis-TEVP fusion protein expression vector, the cis-TEVP expression vector, the cis-TEVP construction vector, the polynucleotide coding for the target protein and/or the suitable host cell, can be provided in the kits, with suitable instructions and other necessary reagents, in order to perform the methods herein disclosed. The kit will normally contain the above components in compositions included in separate containers. Instructions, for example, written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

Further details concerning the identification of the identifier additional component to be included in the compositions, and generally manufacturing and packaging of the kit, can be identified by the person skilled in the art upon reading of the present disclosure.

In another embodiment, production of the recombinant protein is performed following a trans-approach (trans-TEVP-FPS). In the trans-TEVP-FPS, the target protein and a TEVP cleavage of site of sequence SEQ ID NO: 1 are included in a trans-TEVP-FPS fusion protein, while the TEVP function is provided in trans. In the trans-TEVP-FPS upon expression of the trans-TEVP-FPS fusion protein in a suitable host in presence of the TEVP function, site-specific self-cleavage of the trans-TEVP-FPS fusion protein at said TEVP cleavage site, and associated production of the target protein, can be detected.

Preferably the trans-TEVP-FPS fusion protein has the following formula

[X1]-[L]-[X2] (V)

wherein X1, L and X2 have the meaning of formula (I), wherein the TEVP cleavage site included in the L polypeptide is the polypeptide of sequence SEQ ID NO: 1. Various embodiments can be devised, wherein the polypeptides X1, L, and X2 fusion carrier, tag system and linker as disclosed above for the cis-TEVP-FPS.

The trans-FPS and/or the trans-TEVP-FPS fusion protein can be used to produce the target protein in vitro or in vivo, according to procedures herein described and/or identifiable by a person skilled in the art upon reading of the present disclosure and in particular the Example section; those procedures may also include isolation of the fusion protein from the host and/or removal of the target protein from the fusion protein by a variety of enzymatic or chemical means identifiable by a person skilled in the art.

In some embodiments, the trans-TEVP-FPS can be used to produce the target protein following a method comprising: providing a trans-TEVP fusion protein expression vector comprising a polynucleotide encoding for a trans-TEVP fusion protein; providing a TEVP protease expression vector comprising a polynucleotide encoding for a TEV protease; providing a suitable host cell, the host cell transformable by the trans-TEVP fusion protein expression vector and the TEVP protease expression vector; transforming the suitable host cell with the first trans-TEVP fusion protein expression vector and the TEVP protease expression vector; and obtaining the target protein from the transformed cell, the target protein obtained upon expression of the trans-TEVP fusion protein in the suitable host, by site-specific self-cleavage of the trans-TEVP fusion protein at said TEVP cleavage site.

In particular, a trans-TEVP-FPS fusion protein expression vector can be a trans-TEVP-FPS vector which is an expression vector including a trans-TEVP-FPS polynucleotide coding for the trans-TEVP-FPS fusion protein. The trans-TEVP-FPS polynucleotide, preferably has the following formula

[X1′]-[L′]-[X2′] (VI)

wherein, X1′, L′ and X2′ have the same meaning of formula II and the L′ polynucleotide includes the sequence 5′TACGTA3′ in a position such that the sequence 5′TACGTA3′ codes for the amino acid residue in positions P3 and P2 of the TEVP cleavage site.

In other embodiments, the method to produce a target protein with a trans-TEVP-FPS comprises: providing the trans-TEVP fusion protein expression vector; providing a suitable host cell, the host cell able to express a TEV protease and being transformable by the trans-TEVP fusion protein expression vector; transforming the suitable host cell with the trans-TEVP expression vector; and obtaining the target protein from the transformed cell upon expression of the TEV protease, the target protein obtained upon expression of the trans-TEVP fusion protein in the suitable host, by site-specific self-cleavage of the trans-TEVP fusion protein at said TEVP cleavage site.

The trans-TEVP fusion protein expression vector can be obtained from a trans-TEVP expression vector, including a trans-TEVP expression polynucleotide encoding for a trans-TEVP fusion protein comprising a TEVP cleavage site, the amino acid residue in position P2 of the TEVP cleavage site being a Valine, the trans-TEVP expression vector modifiable into the trans-TEVP fusion protein expression vector by introduction in the polynucleotide encoding for a target protein in the TEVP cleavage site, according to methods analogous to the one described for obtaining the cis-TEVP fusion protein expression vector from the cis-TEVP expression vector. Also, the trans-TEVP expression vector can be construed starting from trans-TEVP construction vector, the trans-TEVP construction vector being a vector modifiable into the trans-TEVP expression vector by introduction of a polynucleotide coding for a TEVP cleavage site or portions thereof, according to procedures analogous to the ones described for obtaining the cis-TEVP expression vector from cis-TEVP construction vector.

In some embodiments obtaining a trans-TEVP-fusion protein, a trans-TEVP-fusion protein expression vector, the trans-TEVP expression vector or other proteins or vectors suitable in the trans-approach can be performed according to the sticky-end PCR method herein described with reference to the cis-approach.

Kit of parts for the production of a target protein are also disclosed comprising at least two of the trans-TEVP-FPS fusion protein expression vectors, TEVP protease expression vector, trans-TEVP expression vector, trans-TEVP construction vector and suitable host cell, wherein the host cell is transformed with the trans-TEVP-FPS fusion protein vector and/or the TEVP expression vector according to the methods disclosed herein, the trans-TEVP-FPS fusion protein vector, the TEVP expression vector and the host cell thus enabling the production of the target protein.

In both the cis-approach and trans-approach, the FPS herein disclosed allows efficient protein production in a system that does not necessarily require expensive proteases for fusion protein cleavage and/or tedious cloning efforts of multiple cloning steps performed by using multiple expression vectors.

### EXAMPLES

### Example 1

### Intracellular Processing of MBP-TEVP-RsTEV-GFP-His₆ Fusion Protein

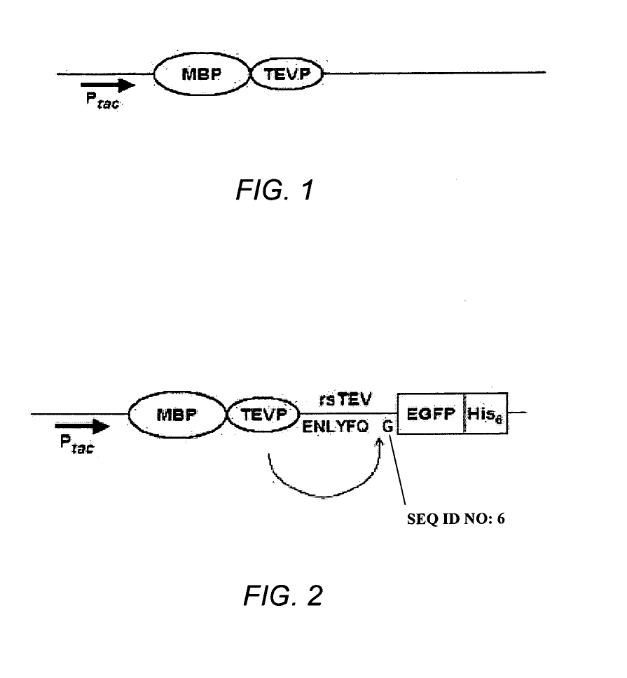

The MBP-TEVP fusion vector schematically shown in FIG. 1 was obtained as described in Shih et al. 2002, and Wang and Wang, 2004.

The MBP-TEVP fusion vector was further modified to include an MBP-TEVP-rsTEV-EGFP-His₆ portion schematically shown in FIG. 2, which is expressed as an MBP-TEVP-rsTEV-EGFP-His₆ fusion protein.

The MBP-TEVP-rsTEV-EGFP-His₆ fusion vector was then cloned in E. coli strain JM109(DE3) and the cells induced with 0.1 mM IPTG at 18-20° C. for 24 hr wherein in log phase (OD₆₀₀˜0.6).

The cells were then harvested and lysed for protein solubility test as described in Shih et al., 2002, and Wang and Wang, 2004, under low induction temperature and long induction time as defined in the two references to facilitate correct protein folding.

The cells were then processed wherein to increase the accuracy of solubility testing, an ultracentrifugal force (90,000 g) was applied to eliminate both partially folded protein aggregates and insoluble materials from total lysates. Hence, proteins in total cell lysates were separated by SDS-PAGE.

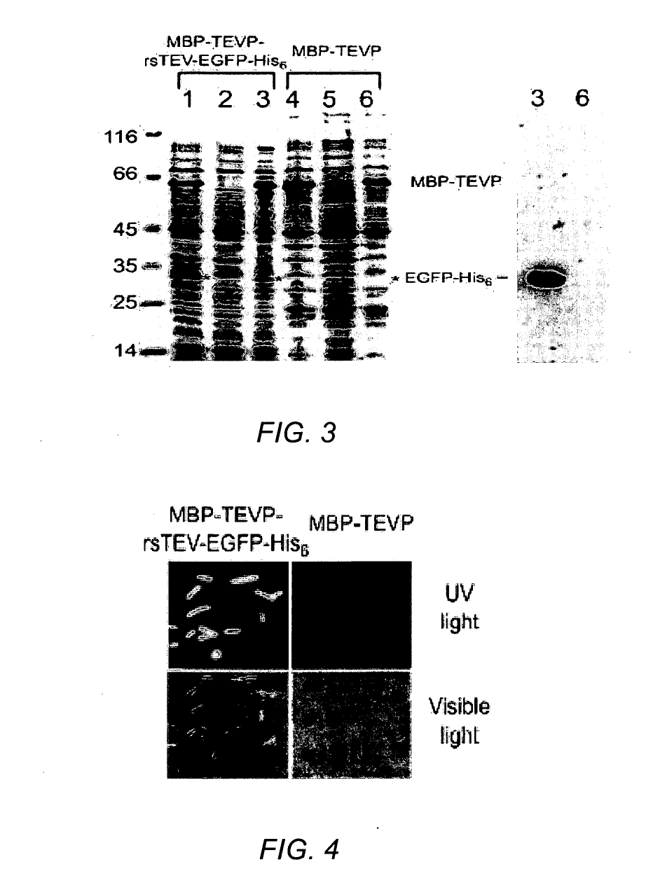

The results are illustrated in FIG. 3A showing SDS page of total cell lysates either from cell induced with IPTG (see FIG. 3A, lanes 1 and 4) or from non-induced cells (see FIG. 3A, lanes 2 and 5), and SDS of soluble protein fraction from IPTG induced cells (FIG. 3A, lanes 3 and 6).

The results shown in FIG. 3 clearly indicate that MBP-TEV fusion protein (apparent molecular weight ˜70,000) was well induced and soluble (see FIG. 3, lane 6) and that the MBP-TEVP-rsTEV-EGFP-His₆ fusion protein was not only well induced but also correctly processed to yield MBP-TEVP-rsTEV and EGFP-His₆, respectively (FIG. 3, lane 3).

Western blotting using anti-His₆ antibody (Clontech, USA) and anti-GFP antibody (Molecular Probes, USA) was then performed to confirm the SDS-PAGE of FIG. 3A.

The results shown in FIG. 3B confirm the expression of and correct processing of the EGFP-His6 protein (FIG. 3B lane 3). Additionally, since almost no signal of unprocessed MBP-TEVP-rsTEV-EGFP-His6 was detected by Western blot using anti-His₆ antibody (FIG. 3B, lane 3), the Western blotting also indicates a yield of intracellular processing near 100%.

To confirm the above results, extracts containing EGFP-His₆ were subjected to purification on Ni²⁺ containing resins that selectively retains His₆-tagged polypeptides (data not shown). Peptide sequencing of the purified protein showed that the NH2-terminal pentamer GEFGL matched the first five amino acid residues of EGFP-His₆.

To further confirm those results, E. coli cells expressing MBP-TEVP-rsTEV-EGFP-His₆ fusion protein were examined by fluorescence microscope together with controls to visualize the EGFP fusion proteins in living cells.

After IPTG induction, E. coli cells were harvested by centrifugation, washed once and then resuspended with the same volume of phosphate-buffered saline. About 2 μl was applied to a microscope slide, excess liquid was aspirated, and a glass cover slip was placed on the slide. The cell outlines were visualized simultaneously with the GFP signal using Chroma filter set no. 86002v1. Images were captured with a Leica DMR microscope plus a cooled charge-coupled device (CCD) camera (Roper Scientific, N.J., USA) and MetaVue software (Universal Imaging Corporation, PA, USA).

The results, illustrated in FIG. 4, show that only cells expressing the MBP-TEVP-rsTEV-EGFP-His₆ fusion protein emitted green fluorescence upon UV light illumination. Taken together, the above experiments support the conclusion that the MBP-TEVP-rsTEV-EGFP-His₆ fusion protein is able to carry out near 100% autonomous site specific processing in vivo.

### Example 2

### Intracellular Processing OF Sso-TEVP-RsTEV-GFP-His₆ Fusion Protein

The experiments described in Example 1 were repeated with the same system described in Example 1 wherein the EGFP protein was replaced by Sulfolobus solfataricus (Sso) 1889 protein.

Accordingly, in the fusion protein expressed by the corresponding modified expression vector the construct EGFP-His₆ was replaced by the construct Sso1889-His₆. The fusion protein modified to include Sso1889-His₆ was cloned and screened as described in Example 1, wherein the proteins in total cell lysates were separated by SDS-PAGE stained by Coomassie blue and subjected to Western Blotting.

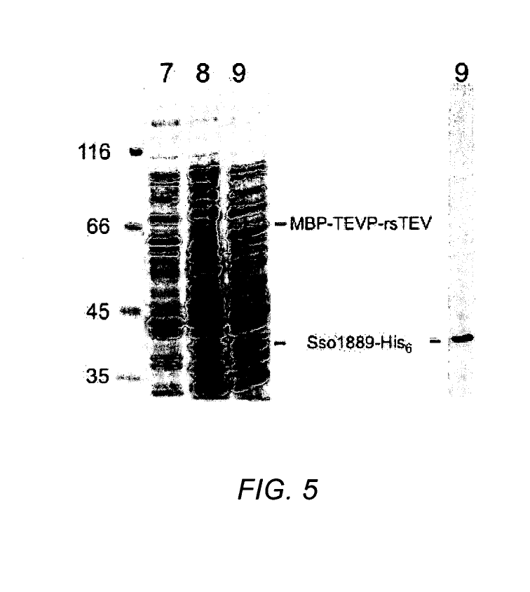

The results illustrated in FIGS. 5A and B, show that MBP-TEVP-rsTEV-Sso1889-His₆ indeed self-cleaved into MBP-TEVP-rsTEV and Sso1889-His₆ (FIG. 5A lane 9 and FIG. 5B lane 9). Also, since MBP-TEVP-rsTEV-Sso1889-His₆ could not be detected by Western blotting using anti-His₆ antibody (FIG. 5B, lane 9), the fusion protein comprising Sso1889-His₆ was completely and specifically cleaved off analogously to what reported for the fusion protein comprising EGFP-His₆.

These results support the conclusion that the self-cleavage of the cis-FPS fusion protein does not depend on the sequence or dimensions of the target protein.

### Example 3

### Production of Recombinant Proteins with Native or Pre-Selected Amino Acid Sequence

In one cloning approach an MBP-TEVP-rsTEV fusion protein vector was provided. The MBP-TEVP-rsTEV fusion protein vector was then modified to introduce an SnaBI and XhoI sites.

A polynucleotide containing the XhoI site and the genetic codons of six Histidine residues were inserted in the pMaI-p2x vector (New England Biolabs, USA) between EcoRI and SalI sites. PmaI-p2x vector expresses a Maltose binding protein (MBP). The resulting vector (pMaI-p2xH) contains the following sequence: 5′ GAATTC [EcoRI]-GGG [Gly]-CTCGAG [XhoI]-(CAC)6 [6xHis]-TAG [stop codon]-GTCGAC [SalI] (SEQ ID NO: 4)

The TEVP cDNA TEVP was inserted into −p2xH by sticky-end PCR cloning method using the EcoRI site. The EcoRI site (GAATTC) at the 5′-end of TEVP cDNA was mutated to GAATTG, so that this vector only contains one EcoRI site immediately at the 3′-end of TEVP clone.

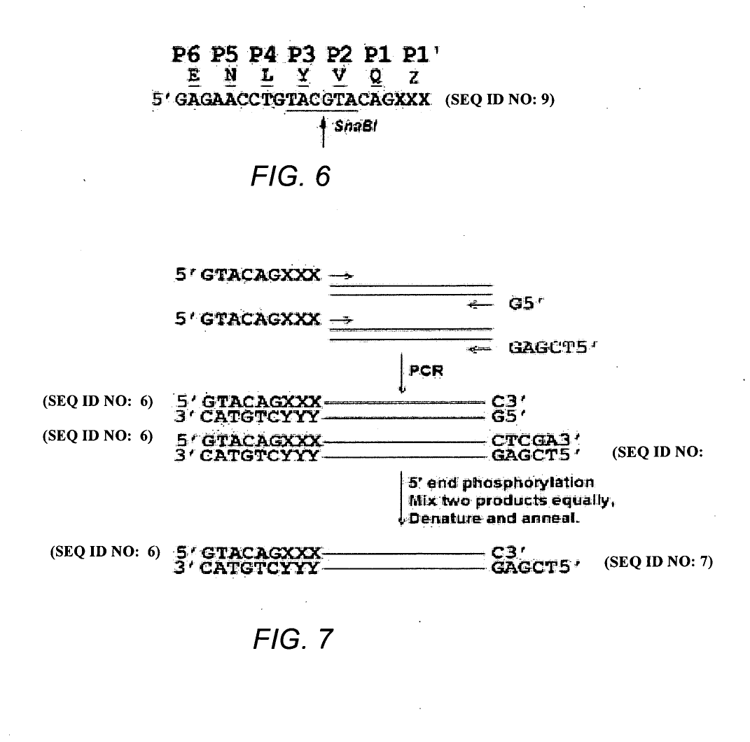

A polynucleotide containing rsTEV, including an SnaBI site as shown in FIG. 6 was inserted between EcoRI and XhoI site of the pMaI-p2xH vector. The SnaBI restriction enzyme site (5′TACGTA3′) was created so that, when translated in the MBP-TEVP-rsTEV-EGFP-His₆ fusion protein vector the Phe residue in the P2 position of the cleavage site (FIG. 1A) will be replaced by a Val residue (see P2 position FIG. 6).

The EGFP was then introduced in the vector including the SnaBI site together with a three nucleotides sequence XXX coding for a pre-selected amino acid residue in P′ position by a cloning strategy comprising two stages.

In the first stage the pre-selected three nucleotide XXX sequence is introduced together with sticky ends matching the XhoI site as schematically shown in FIG. 7. In the second stage, the EGFP is introduced into the vector between the 5′-end SnaBI and 3′-end XhoI sites, resulting in the vector including a MBP-TEVP-rsTEV-EGFP-His₆ fusion protein expressing portion shown in FIG. 8.

In the first stage, the approach schematically illustrated in FIG. 7, was followed. Accordingly, an oligonucleotide having sequence GTACAGXXX was used as a forward primer, wherein the XXX nucleotides constitute the codon of the amino acid residue to be located in P1′ position. In a first reaction the forward primer is used with a first reverse primer. Equal amounts of the two PCR products were then mixed, and the 5′ ends were phosphorylated with T4 polynucleotide kinase. After denaturing (95° C. for 5 min) and renaturing (65° C. for 5 min), ˜50% of the final products carry SnaBI (5′) and XhoI (3′) ends and are ready for ligation to be performed in the second stage even without restriction digestion of PCR products.

In the second stage, the cDNA of EGFP was amplified by PCR from pEGFP-N2 vector (Clontech, USA) and introduced in the vector as described in Shih et al., 2002, and Wang and Wang, 2004. The resulting fusion protein construct is illustrated in FIG. 8, where the amino acid residue pre-selected to be introduced in the P1′ position is generically indicated by the letter Z.

Four different MBP-TEVP-rsTEV-EGFP-His₆ fusion proteins were constructed and expressed in JM109(DE3) strain. Each one of them has different amino acid residues at P1′ position, i.e. Met, Gly, Pro and Val, respectively. Host cells were harvested and lysed, and then subjected for protein solubility test in parallel as reported in Example 1.

The results reported in FIGS. 9A and 9B, show that all these four fusion proteins were effectively processed into MBP-TEVP-rsTEV and EGFP-His₆ in vivo as revealed by both SDS-PGE (9A) and Western blot using anti-GFP antibody (FIG. 9B). Accordingly these results show that FPS wherein the amino acid residue in position P2 is a Val, are able to perform complete site specific cleavage of fusion proteins including Pro, as well as other different amino acid residues in position P1′ (see Example 3 and FIG. 9). These results are surprising in view of previous studies indicated that MBP-rsTEV-NusG with Pro in the P′1 position exhibited no processing in E. coli cells co-expressing TEVP (Kapust et al. 2002).

Overall, these results show the ability of the modified vector to efficiently express proteins with different amino acidic residues at the N-terminus and support the conclusion that the fusion protein expression vector can be used to produce fusion proteins having their native amino terminus.

### Example 4

### Parallel Cloning and Screening of Multiple Self-Cleavage Fusion Protein Vectors

MBP was selected for the experiments as an effective solubilizing agent compared to most other fusion carriers or affinity tags (Shih et al. 1998; Kapust and Waugh, 1999).

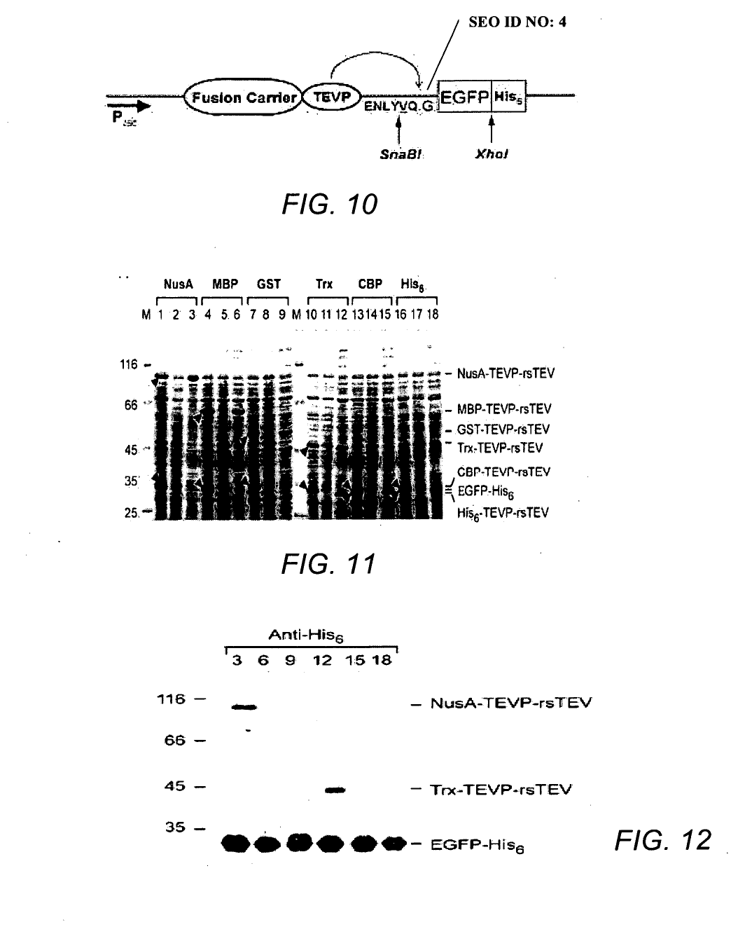

To test the ability of the fusion protein including TEVP and TEVP cleavage site to five additional TEVP fusion vectors were construed similar to the one shown in FIG. 2, wherein the MBP fusion carrier is replaced by other fusion carriers or affinity tag expression systems, such as NusA, thioredoxin (Trx), glutathione S-transferase (GST), or calmodulin binding protein (CBP), His₆ tag, were construed as described in Shih et al., 2002, and Wang and Wang, 2004. These FC-TEVP-rsTEV-EGFP-His₆ vectors shared the same TEV recognition site as well as the SnaBI and XhoI restriction sites according to the schematic representation shown in FIG. 10, wherein the position of each fusion carrier or affinity tag in the vector is indicated by the wording “fusion carrier” FC. Accordingly, the FC-TEVP-rsTEV-EGFP-His₆ fusion vectors can be used for parallel cloning of sticky-end PCR products as described in Example 3 above.

The five fusion vectors including GST-TEVP, Trx-TEVP, NusA-TEVP, CBP-TEVP, His₆-TEVP were construed and subjected to protein solubility tests and Western blotting as reported in Example 1.

The results, illustrated in FIGS. 11 and 12, show that all six vectors successfully carried out intracellular cleavage and produced EGFP-His₆ proteins, as indicated by SDS-PAGE (FIG. 11) and Western blot using anti-GFP antibody (FIG. 12).

The NusA-TEVP-rsTEV and Trx-TEVP-rsTEV band were also recognized by anti-His₆ antibody in the Western blotting analysis, because both NusA and Trx contain an additional His₆ tag (FIG. 12).

These results support the conclusion that processing of the fusion protein in the TEVP cleavage site is independent on the fusion carrier or affinity tag included in the fusion protein.

### Example 5

### Intracellular Processing of MBP-TEVP-RsTEV-GFP-His₆ Fusion Protein

Two vectors were construed following procedures exemplified in Examples 1 and 3 for the expression of a first fusion protein His₆-TEVP and a second fusion protein MBP-rsTEV-EGFP-His₆, respectively. The latter has an ampicillin selection marker, and the former contains a kanamcin selection marker.

When His-TEVP and MBP-reTEV-EGFP-His₆ expression vector was separately transformed into two E. coli host cells, both proteins were produced as expected.

When both expression vectors were transformed into E. coli host cell, the MBP-rsTEV-EFGP-His₆ fusion protein was successfully cleaved into MBP-reTEV and EFGP-His₆.

While the FPSs, fusion proteins, fusion protein vectors, methods and kit of parts have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A cis-TEVP fusion protein system, comprising
a cis-TEVP fusion protein, comprising a TEVP protease and a TEVP cleavage site and
a host cell
wherein site-specific self-cleavage of the cis-TEVP fusion protein at said TEVP cleavage site is detectable upon expression of the cis-TEVP fusion protein in the host cell.

2. A cis-TEVP fusion protein comprising a TEVP protease and a TEVP cleavage site, wherein site-specific self-cleavage of the cis-TEVP fusion protein at said TEVP cleavage site is detectable upon expression of the cis-TEVP fusion protein in the host cell.

3. The cis-TEVP fusion protein of claim 2, having the formula
[X1]-[Y]-[L]-[X2] (I)
wherein, X1 is a polypeptide comprising at least one portion for the expression and/or solubilization of the fusion protein; Y is a polypeptide comprising a TEVP protease; L is a linker polypeptide comprising a TEVP cleavage site; and X2 is a polypeptide comprising at least one target protein.

4. The cis-fusion protein of claim 3, wherein the TEVP cleavage site has a sequence listed in the annexed sequence listing as SEQ ID NO: 1.

5. (canceled)

6. A kit of parts for the production of a target protein, the kit comprising at least two among:
a cis-TEVP fusion protein expression vector, the cis-TEVP fusion protein expression vector including a polynucleotide encoding for a cis-TEVP fusion protein comprising a TEV protease, a TEVP cleavage site and a target protein;
a cis-TEVP expression vector, the cis-TEVP expression vector including a polynucleotide encoding for a fusion protein comprising a TEV protease and a TEVP cleavage site, the cis-TEVP expression vector modifiable into the cis-TEVP fusion protein expression vector by introduction in the polynucleotide encoding for a target protein in the TEVP cleavage site;
a cis-TEVP construction vector, the cis-TEVP construction vector being a vector modifiable into the cis-TEVP expression vector by introduction of a polynucleotide coding for a TEV protease, a TEVP cleavage site or portions thereof; and
a host cell transformable with the cis-TEVP fusion protein expression vector,
the target protein obtained upon transformation of the host cell with the cis-TEVP fusion protein expression vector, by site-specific self-cleavage of the cis-TEVP fusion protein produced in the host cell at said TEVP cleavage site.

7. A trans-TEVP fusion protein system, comprising
a trans-TEVP fusion protein comprising a TEVP cleavage site and a target protein, the amino-terminal portion of the target protein adjacent to the C-terminal portion of the TEVP cleavage site, the amino acidic residue in position P2 of the TEVP cleavage site being a Valine;
a TEVP protease; and
a host cell
wherein site-specific cleavage of said trans-TEVP fusion protein at said cleavage site is detectable, upon expression of the trans-TEVP fusion protein in the host cell in presence of the TEVP protease.

8. A trans-TEVP fusion protein comprising
a TEVP cleavage site and a target protein, the amino-terminal portion of the target protein adjacent to the C-terminal portion of the TEVP cleavage site, the amino acidic residue in position P2 of the TEVP cleavage site being a Valine,
wherein site-specific cleavage of said trans-TEVP fusion protein at said cleavage site is detectable upon expression of the trans-TEVP fusion protein in the host cell in presence of the TEVP protease.

9. The trans-TEVP fusion protein of claim 8, having the formula
[X1]-[L]-[X2] (V)
wherein, X1 is a polypeptide comprising at least one portion for the expression and/or solubilization of the fusion protein; Y is a polypeptide comprising a TEVP protease; L is a linker polypeptide comprising a TEVP cleavage site having sequence listed in the annexed sequence listing as SEQ ID NO: 1; and X2 is a polypeptide comprising at least one target protein.

10. A method to produce a target protein, the method comprising
providing a trans-TEVP fusion protein expression vector comprising a polynucleotide encoding for a trans-TEVP fusion protein, wherein the trans-TEVP fusion protein comprises a TEVP cleavage site and a target protein, with the amino terminal portion of the target protein adjacent to the C-terminal portion of the TEVP cleavage site and the amino acidic residue in position P2 of the TEVP cleavage site being a Valine;
providing a TEVP protease expression vector, the TEVP protease expression vector comprising a polynucleotide encoding for a TEV protease;
providing a suitable host cell, the host cell transformable by the trans-TEVP fusion protein expression vector and the trans-TEVP protease expression vector;
transforming the suitable host cell with the trans-TEVP fusion protein expression vector and the TEVP protease expression vector; and
obtaining the target protein from the transformed cell, the target protein obtained upon expression of the trans-TEVP fusion protein in the suitable host, by site-specific self-cleavage of the trans-TEVP fusion protein at said TEVP cleavage site.

11. A method to produce a target protein, the method comprising:
providing a trans-TEVP fusion protein expression vector comprising a polynucleotide encoding for a trans-TEVP fusion protein, wherein the trans-TEVP fusion protein comprises a TEVP cleavage site and a target protein, with the amino terminal portion of the target protein adjacent to the C-terminal portion of the TEVP cleavage site and the amino acidic residue in position P2 of the TEVP cleavage site being a Valine;
providing a host cell, the host cell able to express a TEV protease and being transformable by the fusion protein expression vector;
transforming the host cell with the trans-TEVP fusion protein expression vector; and
obtaining the target protein from the transformed cell upon expression of the TEV protease, the target protein obtained upon expression of the trans-TEVP fusion protein in the suitable host, by site-specific self-cleavage of the trans-TEVP fusion protein at said TEVP cleavage site.

12. A kit of parts for the production of a target protein, the kit comprising at least two among:
a trans-TEVP fusion protein expression vector including a polynucleotide encoding for a trans-TEVP fusion protein, the trans-TEVP fusion protein comprising a TEVP cleavage site and a target protein, the TEVP cleavage site having a Valine amino acid residue in position P2;
a trans-TEVP expression vector, the trans-TEVP expression vector comprising a TEVP cleavage site, the TEVP cleavage site having a Valine amino acid residue in position P2, the trans-TEVP expression vector modifiable into the trans-TEVP fusion protein expression vector by introduction in the polynucleotide encoding for a target protein in the TEVP cleavage site;
a trans-TEVP construction vector, the trans-TEVP construction vector being a vector modifiable into the trans-TEVP expression vector by introduction of a polynucleotide coding for a TEVP cleavage site or portions thereof, the TEVP cleavage site including a Valine amino acidic residue in position P2; and
a host cell transformable with the trans-TEVP fusion protein expression vector,
wherein the target protein is obtained in the host by site-specific self-cleavage of the trans-TEVP fusion protein at said TEVP cleavage site.

13. A method comprising:
providing a cis-TEVP fusion protein expression vector including a polynucleotide encoding for a cis-TEVP fusion protein, the cis-TEVP fusion protein comprising:
a TEV protease;
a TEVP cleavage site, wherein the TEVP cleavage site corresponds to SEQ ID NO: 2; and
a target protein;
wherein the cis-TEVP fusion protein expression vector is adapted to be transformed in E. coli cells; and
wherein the target protein is obtained by expression of the cis-TEVP fusion protein and site-specific self-cleavage of the cis-TEVP fusion protein at the TEVP cleavage site.

14. The method of claim 13, wherein the TEVP cleavage site corresponds to SEQ ID NO: 3.

15. The method of claim 13, wherein the TEVP cleavage site corresponds to SEQ ID NO: 4.

16. The method of claim 13, wherein the TEVP cleavage site corresponds to SEQ ID NO: 5.

17. A method comprising:
obtaining a cis-TEVP fusion protein expression vector including a polynucleotide encoding for a cis-TEVP fusion protein, the cis-TEVP fusion protein comprising:
a TEV protease;
a TEVP cleavage site, wherein the TEVP cleavage site corresponds to SEQ ID NO: 2; and
a target protein;
transforming E. coli cells with the cis-TEVP fusion protein expression vector; and
obtaining the target protein from the E. coli cells.

18. The method of claim 17, wherein the cis-TEVP fusion protein is obtained by:
obtaining an expression vector having an expression portion, a TEV protease producing gene, and a linker producing a TEV protease cleavage site peptide corresponding to SEQ ID NO: 2;
using a SnaBI restriction enzyme to restrict the expression vector; and
ligating a gene encoding the target protein into the SnaBI restriction site.

19. The method of claim 18, wherein the TEVP cleavage site corresponds to SEQ ID NO: 3.

20. The method of claim 18, wherein the TEVP cleavage site corresponds to SEQ ID NO: 4.

21. The method of claim 18, wherein the TEVP cleavage site corresponds to SEQ ID NO: 5.

22. A method comprising:
in an expression vector having an expression portion, a gene encoding a TEV protease, and a linker producing a TEV protease cleavage site peptide corresponding to SEQ ID NO: 2, using a SnaBI restriction enzyme to restrict the expression vector;
ligating a gene encoding a target protein into the SnaBI restriction site;
transforming E. coli cells with the cis-TEVP fusion protein expression vector; and
obtaining the target protein from the E. coli cells.

23. The method of claim 22, wherein the linker producing the TEV protease cleavage site comprises at least SEQ ID NO: 9.

24. The method of claim 22, wherein the TEVP cleavage site peptide corresponds to SEQ ID NO: 3.

25. The method of claim 22, wherein the TEVP cleavage site peptide corresponds to SEQ ID NO: 4.

26. The method of claim 22, wherein the TEVP cleavage site peptide corresponds to SEQ ID NO: 5.


SEQUENCE LISTING (4 sequences)

SEQ ID NO: 1 - protein, 7 aa, Artificial sequence
  Cleavage site
EXXYVQX

SEQ ID NO: 2 - DNA, 24 bp, Artificial sequence
  Primer sequence
gtacagnnnnnnnnnnnnnnnnnn

SEQ ID NO: 3 - DNA, 26 bp, Artificial sequence
  Primer sequence
tcgagnnnnnnnnnnnnnnnnnnnnn

SEQ ID NO: 4 - DNA, 42 bp, Artificial sequence
  Primer sequence
gaattcgggctcgagcaccaccaccaccaccactaggtcgac